(19) United States
(12) Patent Application Publication
(10) Pub. No.: US 2012/0277541 A1
(43) Pub. Date: Nov. 1, 2012

(54) TWO-PIECE CANNULA, A KIT COMPRISING A TWO-PIECE CANNULA AND AN INSERTER

(76) Inventors: Manoj Bhargava, Thornhill (CA); Shahryar Ahmadi, Little Rock, AR (US)

(21) Appl. No.: 13/518,270

(22) PCT Filed: Dec. 14, 2010
(86) PCT No.: PCT/CA10/01986

§ 371 Date: Jun. 21, 2012

(30) Foreign Application Priority Data
Jan. 4, 2010 (CA) 2689765

(51) Int. Cl. A61B 1/32; A61B 17/34

(52) U.S. Cl. 600/210; 604/164.11

(57) ABSTRACT

The invention relates to a two-piece cannula. The two-piece cannula comprises an outer cannula member that is engageable with an inner cannula member to define a continuous passage when the two cannula members are coupled together. When inserted in body tissue, the continuous passage can receive a surgical instrument, such as an arthroscope, for medically analyzing and/or treating a region of interest located beneath the body tissue. The outer cannula member and the inner cannula member comprise an outer portion and an inner flange, respectively, for sealing and securing the body tissue between the outer portion and the inner flange. In a further embodiment, the invention relates to kit comprising a two-piece cannula and an inserter for inserting the two-piece cannula into body tissue. In yet a further embodiment of the invention, a method is provided for inserting the two-piece cannula into body tissue.

### FIELD

This invention relates to a two-piece cannula that is insertable through body tissue to provide an open passageway through the tissue, preferably for permitting the working end of a surgical instrument to pass therethrough. This invention also relates to a kit for inserting the cannula in tissue. This invention also relates to a method for inserting the cannula in tissue.

### INTRODUCTION

Arthroscopy is a minimally invasive surgical process procedure. It is commonly performed to evaluate and treat joints or internal space pathology. A small incision is made in a patient's tissue, and a cannula is placed through this incision. A cannula is typically a “tube-like” structure that, when inserted into the tissue, provides the surgeon with an open channel between the atmosphere and region of interest located beneath the tissue. The surgeon can then access the region of interest by inserting a medical instrument (such as an arthroscope) through the open passageway created by the cannula.

Various types of cannulas are known. One-piece cannulas that define a channel between the atmosphere and a joint are known in the art.

Two-piece cannulas or “cannula-like” devices are also known in the art. See for example U.S. Pat. Nos. 6,210,397; 6,296,657; 6,663,655; and 7,172,574.

### SUMMARY

In accordance with one aspect of this disclosure, a simplified two-piece cannula is provided. The two-piece cannula has an inner cannula member and an outer cannula member. The inner cannula member is inserted into the tissue and comprises a flange to form a seal with the tissue and a shaft that extends outwardly from the flange. The outer cannula member is releasably engageable with the inner cannula member. The flange of the inner cannula member may be flexible. However, it is fixed in position with respect to the shaft. For example, the flange may comprise a frame that is integrally molded with the shaft or which may be separately formed and then secured to the shaft such as by welding, an adhesive or the like to form a one-piece assembly. Accordingly, the orientation of the flange is fixed with respect to the shaft unlike embodiments of U.S. Pat. No. 6,210,397, which have moving parts. Accordingly, when the inner cannula member is inserted into the tissue and the outer cannula member is engaged with the inner cannula member, such as by screwing the inner and outer cannula members together, the inner cannula member is drawn against the inner surface of the tissue such that the flange independently engages the tissue of the patient. Interaction of the outer cannula member to reorient or move the flange is not required.

An advantage of this design is that a simplified design for a two-piece cannula is provided. Further, the design is robust and may reliably form a seal since no moving parts are required. Further, the use of two members that are interengageable permits a single sized cannula to be used in different patients and surgeries since the length of the passage through the tissue that is provided by the cannula may be adjusted by the degree to which the inner and outer cannula members are interengaged, e.g., screwed together. The length of the cannula can be adjusted by screwing the two pieces to each other so that the overall length of the cannula can be shortened or lengthened to the optimal length for tissue thickness. Accordingly, if a shorter passage is required, then the inner and outer cannula members may be screwed together more then if a longer passage is required.

A further advantage is that the inner and outer margins (e.g., the flanges) can be compressed against the tissues. Preferably, at least the inner margin or flange is made of a soft plastic, that will be pushed over underlying tissue from the inside by intra-articular pressure. The plastic used in the construction of the inner flange is preferably sufficiently flexible such that, upon insertion, the radially outer portion of the inner flange will curl or bend upwardly towards the shaft so as to reduce the radial diameter of the inner flange. However, the plastic from which the inner flange is constructed is preferably sufficiently rigid so as to maintain a seal with the tissue that it abuts when the cannula is assembled.

This has several advantages. For example, it prevents encroachment of soft tissue into the working field, and thereby maximizes visualization. It secures the cannula to the inner tissue and prevents it from coming out. It minimizes fluid leak to soft tissue and prevents swelling. Finally, it gives a surgeon the shortest possible length through a cannula for optimal use of the length of instrument and maximum excursion of the working instrument.

A further advantage is that the inner margin (e.g., flange) will cover the hole beside it, in the situation that the entry point to the joint needs to be changed.

In accordance with another embodiment of this disclosure, a cannula kit is provided. The cannula kit comprises a two-piece cannula and an inserter. An optional piercing device may also be provided. The two-piece cannula has an inner cannula member and an outer cannula member. The inner cannula member is inserted into the tissue. The outer cannula member is releasably engageable with the inner cannula member. The inserter is also releasably engageable with the inner cannula member. Therefore, the inner cannula member may be mounted to the inserter (e.g., by being screwed thereon). The inner cannula member may then be inserted into the tissue. The outer cannula member may be slid along the inserter to a position at which it may be engaged with the inner cannula member (e.g., by being screwed onto a shaft of the inner cannula member). Once the inner and out portions are releasably secured together, the inserter may be removed from the inner cannula member (e.g., by unscrewing the inserter). The two-piece cannula may now be used.

Optionally, a piercing device is provided. It is preferred that the inserter is hollow so that it may be slid over the piercing device. The piercing device may be used to create an incision in the tissue for the cannula. The piercing device may have a depth gauge so that it may be inserted a desired depth into the tissue. The inserter may then be slid along the piercing device to insert the inner cannula member of the cannula. The outer cannula member of the cannula may then be secured to the inner cannula member and the inserter and piercing member removed.

An advantage of this design is that a simplified design for a kit to insert a two-piece cannula is provided. Further, the design is robust and may reliably insert the cannula with reduced stress to the tissue thereby accelerating the recovery of the patient.

Accordingly, in accordance with this invention there is provided a cannula insertable through tissue, the cannula comprising:
(a) an outer cannula member that is engageable with an inner cannula member;
(b) the outer cannula member having an outer portion positionable in abutting relationship with an outer tissue surface of the tissue and having an outer instrument opening, and a first shaft with a first passage aligned with the outer instrument opening; and,
(c) the inner cannula member having an inner flange positionable in abutting relationship with an inner tissue surface of the tissue and having an inner instrument opening, and a second shaft with a second passage aligned with the inner instrument opening, the inner flange being fixedly mounted in position with respect to the second shaft and independently engageable in abutting relationship with the inner tissue surface when the inner and outer cannula members are engaged;
   whereby the first and second passages define a continuous passage when the inner and outer cannula members are engaged.

In any embodiment, the second shaft may have a first end and a distal end spaced from the inner flange and the first shaft may be configured to terminate at a location on the second shaft spaced from the first end of the second shaft when the inner and outer cannula members are engaged.

In any embodiment, the second shaft may be engagably received in the first shaft. Preferably, the first shaft has an inner surface having a first engagement member and the second shaft has an outer surface having a second engagement member. Preferably, the first and second engagement members comprise interengaging screw threads.

In any embodiment, the second shaft may have a first end and a distal end spaced from the inner flange and the inner flange may be located adjacent the first end.

In any embodiment, the inner flange may be an annular flange extending around the perimeter of the second shaft. Preferably, the inner flange has an inner flange tissue-facing surface positioned to face the inner tissue surface, the inner flange tissue-facing surface is concave.

In any embodiment, the outer portion may further comprise at least one biological matter vent spaced from the outer instrument opening.

In any embodiment, the outer portion may further comprise an outer portion tissue facing surface and the first shaft may terminate proximate the outer portion tissue facing surface.

In any embodiment, the outer portion may comprise an annular flange extending around the first shaft. Preferably, the outer portion has an outer portion tissue facing surface that is concave.

In any embodiment, at least one of the outer portion and the inner flange may comprise a frame portion and a membrane portion and the membrane portion may have increased flexibility compared to the frame portion.

In any embodiment, the inner cannula member may have an inserter-engaging member.

In accordance with this invention there is also provided a cannula kit including a cannula insertable through tissue, the kit comprising:
(a) an outer cannula member that is engageable with an inner cannula member;
(b) the outer cannula member having an outer portion positionable in abutting relationship with an outer tissue surface of the tissue and having an outer instrument opening, and a first shaft with a first passage aligned with the outer instrument opening;
(c) the inner cannula member having an inner flange positionable in abutting relationship with an inner tissue surface of the tissue and having an inner instrument opening, and a second shaft with a second passage aligned with the inner instrument opening, and an inserter-engaging member; and,
(d) an inserter engageable with the inserter-engaging member.

In any embodiment, the inserter may comprise a longitudinally extending member having a hand grip portion and an insertion end spaced therefrom, the inserter may have a cannula-engaging member provided on the insertion end and releasably engageable with the inner cannula member. Preferably, the first shaft has an inner surface having a first engagement member and the second shaft has an outer surface having a second engagement member and an inner surface having the inserter-engaging member. Preferably, the first and second engagement members comprise interengaging screw threads. Alternately, or in addition, the inserter-engaging member may comprise a screw thread and the insertion end is threadedly engageable therewith.

In any embodiment, the second shaft may have a first end and a distal end spaced from the inner flange and the inner flange may be located adjacent the first end.

In any embodiment, the outer cannula member may be slidably receivable on the inserter.

In any embodiment, the kit may further comprise a piercing device for creating an incision in the tissue, and the piercing device has a plurality of insertion depth markings disposed along its length. Preferably, the inserter further comprises an inserter passage sized to receive therein the piercing device.

In any embodiment, the inserter may further comprise an inserter passage sized to slidably receive therein a longitudinally extending piercing device, the piercing device may have a length longer than the inserter.

In accordance with this invention there is also provided a method for inserting a cannula in a tissue comprising:
(a) mounting an outer cannula member to an inserter and securing an inner cannula member to an insertion end of the inserter;
(b) advancing a piercing device a desired distance into the tissue and positioning the piercing device through the inserter;
(c) advancing the inserter along the piercing device into the tissue until at least a portion of the inner cannula member that is located at the insertion end of the inserter passes an inner tissue surface of the tissue;
(d) advancing the outer cannula member along the inserter toward the insertion end of the inserter and coupling the outer cannula member to the inner cannula member;
(e) detaching the inserter from the inner cannula member; and,
(f) removing the inserter and the piercing device from the tissue.

In any embodiment, the outer cannula member may comprise a first shaft with a first passage aligned with an outer instrument opening, the inserter may be slidably receivable in the first passage, the inner cannula member may be rotatably mountable to the inserter and step (a) may comprise rotatably mounting the inner cannula member to the inserter and sliding the outer cannula member onto the inserter. Preferably, the inserter comprises an inserter passage, the piercing member is slidably receivable in the inserter passage and step (b) comprises sliding the inserter onto the piercing device. Preferably, the inner and outer cannula members are rotatably mountable to each other and step (e) comprises sliding the outer cannula member along the inserter to engage the inner cannula member and then rotatably coupling the inner and outer cannula members. Alternately, or in addition, step (f) comprises rotatably decoupling the inserter and the inner cannula member while maintaining the inner and outer cannula members in a coupled state.

### DRAWINGS

The various embodiments and advantages will be more fully understood in accordance with the following description of the preferred embodiments of the invention in which:

FIG. 1 is a perspective view of a cannula in accordance with this invention comprising an outer cannula member and an inner cannula member, when the two cannula members are disengaged from each other;

FIG. 2 is a cross sectional view along the line 2-2 in FIG. 1 when the two cannula members are engaged with each other;

FIG. 3 is a perspective view of the cannula of FIG. 1 when the two cannula members are engaged with each other and are position in tissue;

FIGS. 4-6 are perspective views of a kit comprising a two-piece cannula and an inserter in accordance with another embodiment of the invention, showing the steps of preparing the kit for use to insert the cannula in a patient;

FIG. 7 is a perspective view of a piercing device that may be included in the kit of FIG. 4, wherein the piercing device is inserted into the tissue; and,

FIGS. 8-10 are perspective views showing the assembly of FIG. 6 being slid along the piercing device of FIG. 7 so as to insert the inner cannula member into the tissue and to engage the inner and outer cannula members.

### DESCRIPTION OF VARIOUS EMBODIMENTS

Various apparatuses or methods will be described below to provide an example of each claimed invention. No invention described below limits any claimed invention and any claimed invention may cover processes or apparatuses that are not described below. The claimed inventions are not limited to apparatuses or processes having all of the features of any one apparatus or process described below, or to features common to multiple or all of the apparatuses described below. It is possible that an apparatus or process described below is not an embodiment of any claimed inventions.

The cannula may be utilized in treating any animal, and preferably a person. It is particularly preferred for providing a surgical passage to access a joint of a person.

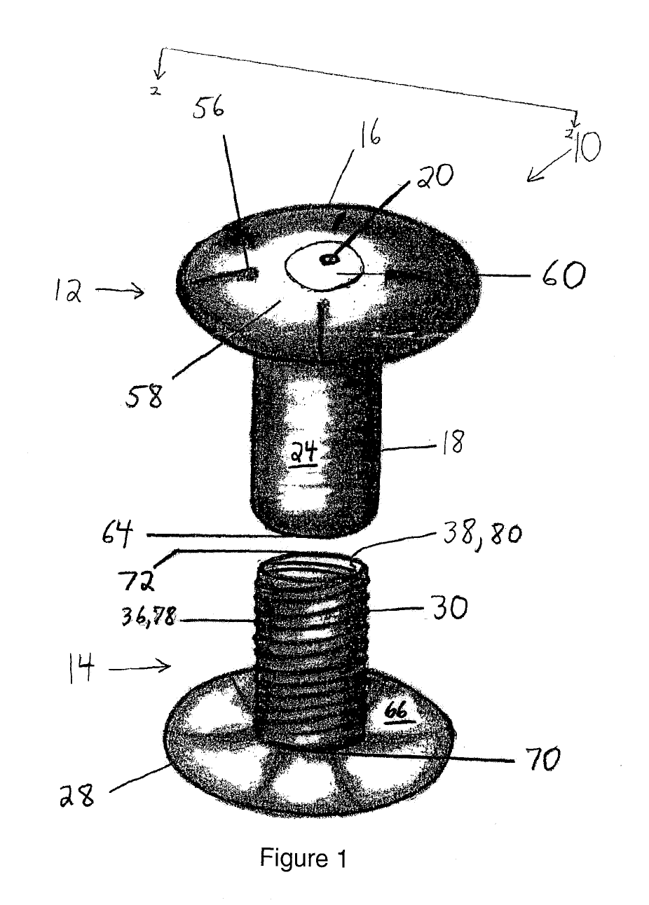

As exemplified in FIG. 1, cannula 10 comprises an outer cannula member 12 and an inner cannula member 14.

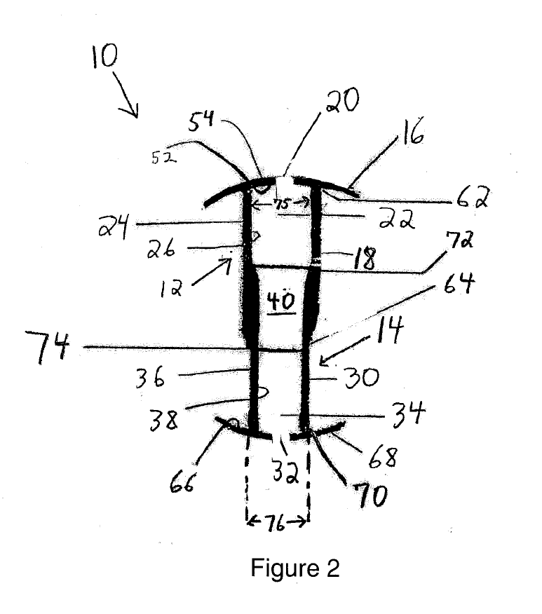

Outer cannula member 12 comprises an outer portion 16 and a first shaft 18 having a first shaft first end 62 and a first shaft distal end 64. The outer portion has an outer instrument opening 20. Preferably, outer instrument opening 20 is centrally located on outer portion 16. Outer instrument opening 20 may receive a surgical instrument such as, but not limited to, a scope such as an arthroscope, surgical instruments, sutures and implants. First shaft 18 has a first passage 22 (FIG. 2). First shaft 18 comprises a first shaft outer surface 24 and a first shaft inner surface 26 (illustrated in FIG. 2). First shaft inner surface 26 encloses a hollow space defining first passage 22. Outer portion 16 is coupled to first shaft 18 such that outer instrument opening 20 aligns with first passage 22. Due to the alignment of the outer instrument opening and the first passage, the working end of a surgical instrument may be received therethrough. It will be appreciated that only a portion of outer instrument opening 20 and first passage 22 need to align with one another to receive a surgical instrument therethrough.

As illustrated in FIG. 1, inner cannula member 14 comprises an inner flange 28 having a radially outer portion 112 and a second shaft 30 having a second shaft first end 70 and a second shaft distal end 72. The inner flange has an inner instrument opening 32 (illustrated in FIG. 2). Preferably, inner instrument opening is centrally located on inner flange 28. As illustrated in FIGS. 1 and 2, second shaft 30 has a second passage 34. Second shaft 30 comprises a second shaft outer surface 36 and a second shaft inner surface 38. Second shaft inner surface 38 encloses a hollow space defining second passage 34. Inner flange 28 is coupled to second shaft 30 such that inner instrument opening 32 aligns with second passage 34. Due to the alignment of the inner instrument opening and the second passage, the working end of a surgical instrument may be received therethrough.

Outer cannula member 12 is engageable, and preferably releasably engageable, with inner cannula member 14. As exemplified in FIG. 2 when engaged, the two cannula members 12, 14 define a continuous passage 40. Continuous passage 40 comprises first passage 22 and second passage 34. Therefore, the working end of a surgical instrument may be inserted through outer instrument opening 20, through continuous passage 40 and exit cannula 10 via inner instrument opening 32.

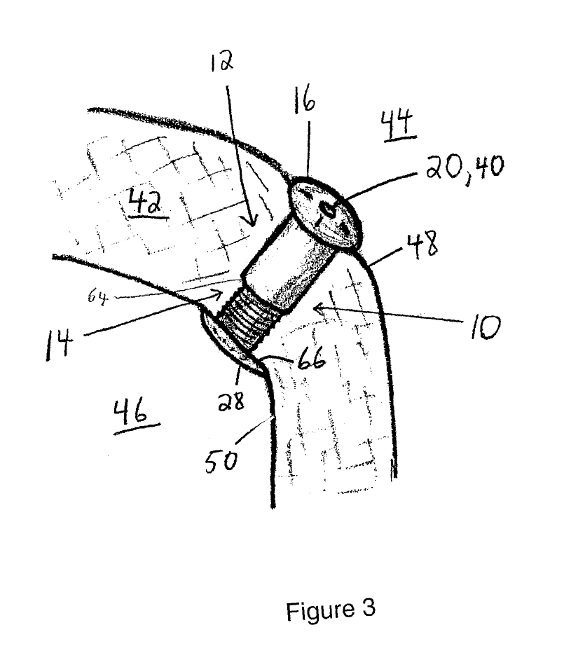

FIG. 3 illustrates cannula 10 inserted into tissue 42, with outer cannula member 12 coupled with inner cannula member 14 and continuous passage 40 extending from the external atmosphere 44 to an internal joint region 46. Tissue 42 has an outer tissue surface 48 and an inner tissue surface 50. As exemplified, outer portion 16 is positioned in abutting relationship with outer tissue surface 48 and inner flange 28 is positioned in abutting relationship with inner tissue surface 50. Accordingly, a surgeon may access internal joint (or any internal space) region 46, for example, by inserting the working end of a surgical instrument (such as an arthroscope) through continuous passage 40 (FIG. 2).

Preferably, outer cannula member 12 is adjustably engageable with inner cannula member 14. As a result, the distance between outer portion 16 and inner flange 28, when outer cannula member 12 is coupled with inner cannula member 14, may be varied. It will be appreciated that decreasing the distance between outer portion 16 and inner flange 28 will compress the tissue between the outer portion and the inner flange 28. Outer cannula 12 and inner cannula member 14 may be adjustably engaged to the point that outer portion 16 and inner flange 28 are at a specific distance away from one another. Preferably, this specific (possibly predetermined) distance causes a compressive force to be exerted on tissue 42. The greater the compressive force, the better the seal that will be formed between flange 28 and inner tissue surface 50 and the seal between outer portion 16 and outer tissue surface 48. Another advantage of outer cannula member 12 being adjustably engageable with inner cannula member 14 is that the length of cannula 10 can be increased or decreased to accommodate tissues of varying thicknesses. In addition, tissue 42 may swell in reaction to the insertion of cannula 10. The length of cannula 10 may be adjustably increased to accommodate this swelling action.

Outer portion 16 will now be discussed in detail. The outer portion may be any member that will engage the outer tissue surface 48 and resist outer portion 16 being drawn through an incision in tissue 42 as the inner and outer cannula members 12, 14 are secured together. For example, outer portion 16 may have a plurality of legs extending out from first shaft 18.

Preferably, outer portion 16 is configured to create a seal between outer portion 16 and outer tissue surface 48. Accordingly, biological matter (e.g., fluids) that might otherwise flow out of the incision in the tissue may be prevented from flowing along outer tissue surface 48 in the area of the incision and contaminating the external surroundings. Further, the seal reduces or prevents encroachment of soft tissue into the working field, and thereby maximizes visualization.

As exemplified in FIG. 1, outer portion 16 may comprise a flange. Preferably, outer portion 16 comprises an annular flange that extends around the perimeter of first shaft 18, wherein the perimeter of first shaft 18 is defined by first shaft outer surface 24. As exemplified in FIG. 2, outer portion 16 comprises an outer flange tissue-facing surface 52 and an outer portion outer surface 54. The outer flange tissue-facing surface 52 is positionable to face outer tissue surface 48 (FIG. 3). Outer surface 54 may substantially oppose outer flange-tissue facing surface 52. Preferably, when outer portion 16 is in abutting relationship with outer tissue surface 48, outer flange tissue-facing surface is in contact with outer tissue surface 48 so as to create a seal.

Preferably, outer flange tissue-facing surface 52, and optionally outer portion 16, is concave (see FIG. 2). The concave shape may create a suction effect between outer flange tissue-facing surface 52 and outer tissue surface 48 (FIG. 3). This suction effect may allow outer portion 16 to better seal the portion of outer tissue surface 48 that abuts outer flange tissue-facing surface 52 and may increase the degree to which cannula 10 is secured to tissue 42.

Preferably, as exemplified in FIGS. 1 and 2, first shaft 18 extends inwardly from outer portion 16. Preferably, first shaft 18 terminates proximate the outer portion tissue-facing surface 54, e.g., first shaft first end 62 is located at the outer portion tissue-facing surface 54. Accordingly, passage 22 does not extend upwardly past outer portion outer surface 54 so as to restrict the motion of surgical instruments in passage 22.

As exemplified in FIG. 1, in any embodiment, outer portion 16 may comprise one or more biological matter vent 56. The biological matter vents 56 provide an opening in the outer portion through which biological matter (e.g., fluid) may flow to exit the incision. Preferably, biological matter vents 56 are provided in the portion of outer portion 16 that extends outwards from first shaft 18. Preferably, outer portion 16 comprises a plurality of biological matter vents 56. Preferably, each biological matter vent 56 is in the shape of a slot that extends radially outward from outer instrument opening 20.

Accordingly, fluid may flow outwardly in the space between first shaft 18 and tissue 42 and be evacuated via biological matter vents 56. A suction line or the like may be connectable (e.g., insertable) into biological matter vents 56 so as to draw away fluid and prevent the fluid from contaminating the work area. For example, vents 56 may control the flow and/or drain fluid from the joint or inner space at the end of the procedure or even during the procedure. As an example, the fluid in the joint may be bloody and unclear and therefore block the working field. This bloody fluid may be drained and optionally replaced with clear fluid.

First shaft 18 is preferably sufficiently rigid to maintain passage 22 open and to resist outer cannula member 12 being pushed out of the incision. For example, shaft 18 may be constructed from plastics or metals or combination of them and more preferably plastics. Outer portion 16 may be made of the same material. Accordingly outer portion 16 may be integrally molded with shaft 18.

Alternately, it is preferred that at least a portion of outer portion 16 is flexible, particularly in the vicinity of outer instrument opening 20. For example, when a surgical instrument is inserted into outer instrument opening 20, the opening can be stretch to accommodate the instrument and minimize gaps between the perimeter of the opening and the instrument. This stretching action may create a seal between outer portion 16 and the surgical instrument. Accordingly, the amount of biological matter that can escape from potential gaps formed between the surgical instrument and the flexible membrane portion is minimized. Accordingly outer portion 16 may be made from a resilient or yieldable material such as plastics or metals or combination of them and preferably plastics. Outer portion 16 may be secured to shaft 18 such as by welding, an adhesive, a compression fit or the like.

In a particularly preferred embodiment, outer portion 16 comprises a relatively rigid frame portion 58 and a relatively flexible membrane portion 60 (see FIG. 2). If vents 56 are provided, they are preferably provided on rigid frame portion 58. Membrane portion 60 may define outer instrument opening 20. Frame portion 58 may be of any configuration that provides a base for membrane 60 and may be made of any material that is more rigid then membrane 60. For example, rigid frame portion 58 may be made of PET. The plastic used in the construction of the frame is preferably sufficiently rigid so as to maintain a seal with the tissue that it abuts when the cannula is assembled. The frame may comprise a plurality of outwardly extending legs and may optionally have an outer annular band, so as to resemble the frame of a wheel for a bicycle. This configuration permits the outer portion to seal against tissue 42 yet provide a flexible portion around opening 20. Frame portion 58 may be made of the same material as shaft 18 and may be integrally molded therewith. Alternately, frame portion 58 may be secured thereto by welding, an adhesive, a compression fit or the like. Membrane 60 may be made of any yieldable or resilient material. Preferably, membrane 60 is made from an elastomeric material. Membrane 60 is preferably sufficiently flexible such that is may form a seal around an instrument inserted into passage 22. Membrane 60 may be provided on frame portion 58 such as by over molding.

Inner flange 28 will now be discussed in detail. Inner flange 28 may be of any construction discussed with respect to outer portion 16. Inner flange 28 comprises an inner flange tissue-facing surface 66 and an inner flange inner surface 68. The inner flange tissue-facing surface 66 is positionable to face inner tissue surface 50 (see FIG. 3). Inner flange inner surface 68 may substantially oppose inner flange-tissue facing surface 66. Preferably, when inner flange 28 is in abutting relationship with inner tissue surface 50 (as illustrated in FIG. 3), at least a portion, and preferably an annular band of inner flange tissue-facing surface 66 is in contact with inner tissue surface 50. Accordingly, as exemplified in FIG. 3, inner flange 28 may act as a seal for inner tissue surface 50. Biological tissue matter that could otherwise flow from the work area in tissue 42 may be prevented from seeping out between outer surface 36 of second shaft 30 and tissue 42. Further, inner flange 28 may reduce or prevent the encroachment of soft tissue into the working field, and thereby maximizes visualization. Further, inner flange 28 may secure the cannula to the inner tissue and prevents it from coming out.

In a preferred embodiment, inner flange 28 is independently engageable in abutting relationship with inner surface tissue 50. That is, when inner cannula member 14 is drawn against inner tissue surface 50, inner flange 28 creates a seal against inner tissue surface 50 without the inner flange being moved or deformed by an external member, such as outer cannula member 12. The position of inner cannula member 14 relative to inner tissue surface 50 dictates whether inner flange 28 and inner tissue surface 50 are in sealing relationship with one another. In this particularly preferred embodiment, inner flange 28 preferably comprises an annular flange that extends around second shaft 30. Alternately, or in addition, inner flange tissue-facing surface 66 is concave. The concave shape may create a suction effect between inner flange tissue-facing surface 66 and inner tissue surface 50. This suction effect can allow inner flange 28 to more efficiently seal a portion of inner tissue surface 50 that abuts inner flange tissue-facing surface 66 and can increase the degree to which cannula 10 is secured to tissue 42.

As with outer cannula member 12, inner flange 28 may comprise a frame portion 58 and a membrane 60.

As with outer cannula member 12, second shaft 30 preferably extends inwardly from inner flange 28. Preferably, second shaft 30 terminates proximate to inner flange tissue-facing surface 66.

The engagement of outer cannula member 12 and inner cannula member 14 will now be discussed in detail. Inner and outer cannula members 14, 12 are preferably releasably engageable. Alternately, or in addition, inner and outer cannula members 14, 12 are preferably engageable so as to adjust the length of passage 40. In a particularly preferred embodiment, inner and outer cannula members 14, 12 are preferably rotatably engageable, such as by screw threads or a bayonet mount. Accordingly, first shaft 18 and second shaft 30 may be substantially cylindrical with first shaft 18 having a first shaft cross-sectional diameter 75 bound by first shaft inner surface 26 and second shaft 30 having a cross-sectional diameter 76 bound by the second shaft outer surface 36. If second shaft 30 is received in first shaft 18, which is preferred, then the first shaft cross-sectional diameter is larger than the second shaft cross-sectional diameter, such that the first shaft 18 can receive at least a portion of second shaft 30.

Referring to FIG. 1, outer cannula member 12 and inner cannula member 14 are preferably engaged by coupling first shaft 18 of the outer cannula member to second shaft 30 of the inner cannula member. When the outer and inner cannula members are engaged, distal end 64 of first shaft 18 may be located spaced from first end 70 of second shaft 30, such as at an intermediary location 74 on second shaft 30.

First shaft 18 may comprise a first engagement member 110 and second shaft 30 may comprise a second engagement member 78. First engagement member 110 and second engagement member 78 may cooperate with one another to secure outer cannula member 12 to inner cannula member 14. The first and second engagement members may be any members known in the fastener art that permits two members to interengage.

Preferably, outer cannula member 12 and inner cannula member 14 may be engaged with one another so as to adjust the length of passage 40. Alternately, or in addition, outer cannula member 12 and inner cannula member 14 may be releasably engaged with one another.

As exemplified in FIGS. 1 and 2, first engagement member 110 may be provided on shaft 18 and is preferably provided on first shaft inner surface 26 and second engagement member 78 may be provided on shaft 30 and is preferably provided on second shaft outer surface 36. As illustrated, first and second engagement members 110, 78 may comprise interengaging screw threads. Through the cooperation of the complimentary screw threads, outer cannula member 12 and inner cannula member 14 can be engaged with one another. It will be appreciated that by rotating one of the inner and outer cannula members with respect to the other, the distance between outer portion 16 and inner flange 28 can be increased or decreased, thereby adjusting the length of passage 40.

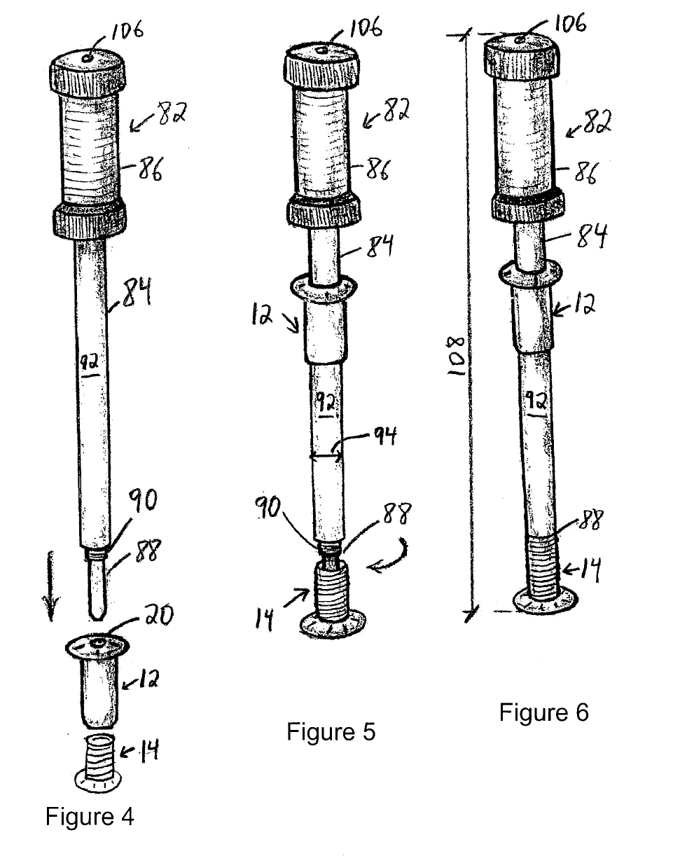

In another aspect, an inserter 82 may be provided as part of a kit. An inserter is a tool that is typically used to insert a cannula partially through tissue 42. Usually, the user exerts a manual push force on the inserter to insert a cannula at least partially into tissue 42. As exemplified in FIG. 4, inserter 82 comprises a longitudinally extending member 84 (e.g., a shaft, which is preferably hollow) having an insertion end 88 having a cannula engaging member 90 and an opposed end that may have a hand grip portion 86. The portion of shaft 84 extending away from hand grip portion 86 preferably has a length at least as long as inner cannula member 4, outer cannula member 12 and the thickness of tissue 42. Insertion end 88 is configured to be insertable through tissue 42. Typically, the inserter is pushed through the tissue by manual force. The hand grip portion 86 may be configured to be easily grabbed by a user's hand. As the user grabs hand grip portion 86 and pushes it toward tissue 42, inserter 82 can be advanced at least partially through tissue 42.

In this aspect, the inserter is engageable with cannula 10 to insert cannula 10 into an incision. Preferably, inserter 82 is engageable with inner cannula member 14. For example, as exemplified in FIG. 1, inner cannula member 14 comprises an inserter-engaging member 80. The inserter-engaging member 80 is configured to couple inner cannula member 14 to inserter 82. Preferably, inserter 82 is rotatably engageable with inner cannula member 14.

For example, mating screw threads of a bayonet mount may be used. Accordingly, as exemplified in FIGS. 1 and 4, inner cannula member 14 may be provided with screw threads located on inner surface 38 of second shaft 30 and cannula engaging member 90 may comprise mating screw threads. Therefore, rotating one of either inner cannula member 14 or inserter 82 engages the members together.

As will be appreciated, it is preferred that second engagement member 78 and inserter engaging member 80 are provided on opposite surfaces of shaft 30. In the exemplified embodiment, the inserter engaging member 80 is provided on inner surface 38 and second engagement member 78 is provided on outer surface 36. Accordingly, outer cannula member 12 may be secured (e.g., screwed onto) inner cannula member 14 while inserter 82 is secured to inner cannula member 14. Therefore, inserter 82 may be used to hold inner cannula member in place while cannula 10 is assembled in situ. Preferably, if inserter engaging member 80 and second engagement member 78 are screw threads, they have the opposite threading so that rotation of the inner cannula member 12 during assembly of cannula 10 will not result in inserter 82 being unscrewed from inner cannula member 12.

A preferred assembly of the inner and outer cannula members and inserter 82 in preparation for the insertion of the cannula 10 into tissue 42 is exemplified in FIGS. 4-6. FIG. 4 exemplifies the elements in an exploded view in preparation for assembly. FIG. 5 illustrates outer cannula member 12 having been slid onto shaft 84 of inserter 82. FIG. 6 illustrates inner cannula member 14 engaged with inserter 82, after one of the inner cannula member and the inserter has been sufficiently rotated in an engaging direction. By rotating one of the inner cannula member or the inserter in an opposite direction to the engaging rotation direction, inner cannula member 14 and inserter 82 can be disengaged.

Outer cannula member 12 is moveably mounted on shaft 84. Preferably, outer cannula member 12 is slidably receivable on shaft 84 of inserter 82. In one embodiment, first shaft cross-sectional diameter 75 may be larger than extended member cross section diameter 94 (see FIG. 5), so first passage 22 of outer cannula member 12 can receive shaft 84 of inserter 82. Similarly, the diameter of outer instrument opening 20 may be (or can at least flex to be) larger than extended member cross section diameter 94. As a result, outer cannula member 12 can freely slide along longitudinally extended member 84.

Preferably, outer cannula member 12 and inserter 82 are configured such that outer cannula member 12 will remain in position on inserter 82 while inner cannula member 14 is inserted into tissue 42. Accordingly, outer cannula member 12 is preferably not freely slidable on shaft 84 or is freely slidable but maintained in position, e.g., by engagement with hand grip portion 86 (e.g., by mating screw threads). For example, passage 22 and shaft 84 may be dimensioned so as to have a slight friction fit. Alternately, opening 20 may provide a friction fit with shaft 84.

As illustrated in FIG. 6, outer cannula member 12 and inner cannula member 14 can be simultaneously engaged with inserter 82. Preferably, this simultaneous engagement is achievable by first sliding outer cannula member 12 onto inserter 82 spaced from insertion end 88 and then engaging inner cannula member 14 with insertion end 88 of inserter 82.

In operation, the kit may be assembled as shown in FIG. 6. Inserter 82 may then be used to place inner cannula member 14 into tissue 42. Outer cannula member 12 may be slid along shaft 84 and engaged with inner cannula member 14. Inserter 82 may then be rotated to disengage inserter 82 from inner cannula member 14.

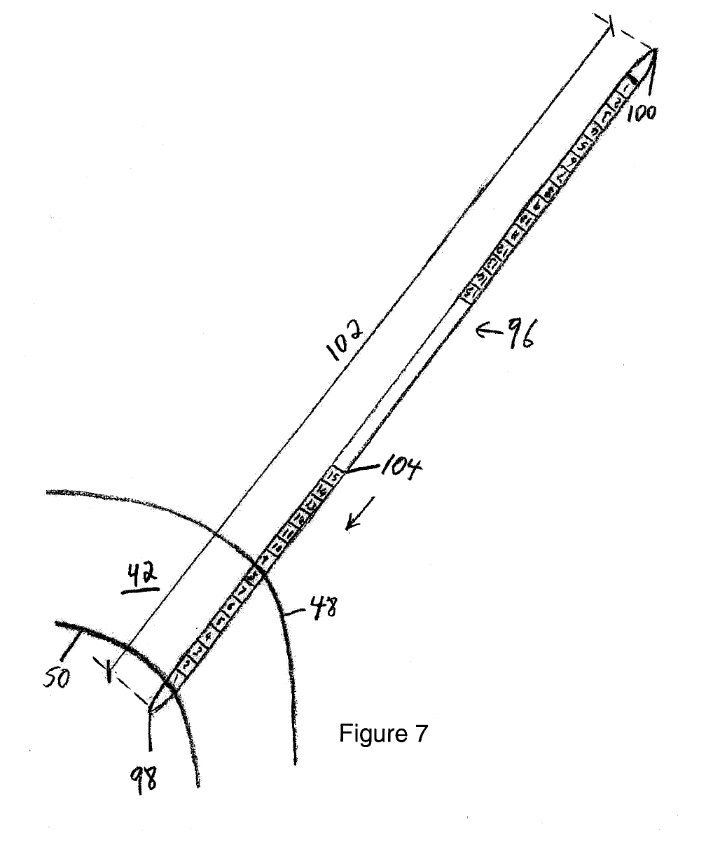

In a further embodiment, the kit comprises an optional piercing device. An exemplary piercing device 96 is illustrated in FIG. 7. A piercing device may be configured to create an incision in tissue 42. Alternately, or in addition, piercing device 96 may be used to guide an inserter, such as inserter 82 (FIG. 6) through the incision created by the piercing device.

As exemplified in FIG. 7, piercing device 96 may be a longitudinally extending member or shaft having a piercing device first end 98 and a piercing device second end 100. The piercing device first end is preferably configured to create an incision in tissue 42 (e.g., it may be pointed).

Piercing device 96 may comprise at least one insertion depth marking 104. Preferably, the piercing device comprises a plurality of insertion depth markings 104 disposed along its length. When the piercing device is inserted into tissue 42, the insertion depth markings can inform the user about tissue depth, as measured between outer tissue surface 48 and inner tissue surface 50. The insertion depth markings can also provide an indication of the distance from outer tissue surface 48 to a joint region of interest. The insertion depth markings may be provided at each end 98, 100 and each end 98, 100 may be pointed.

The piercing device has a piercing device length 102, measured between its first end 98 and second end 100. Length 102 is preferably at least as long as the length of inserter 82 when inner cannula member 14 is mounted thereto.

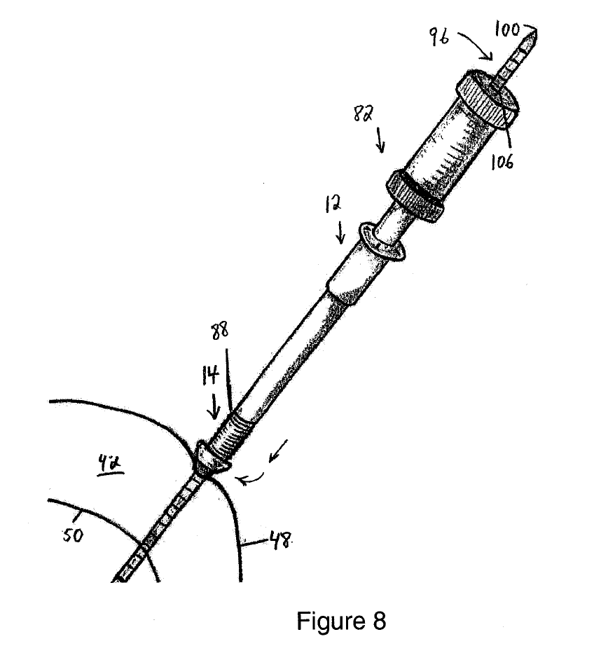

Preferably, inserter 82 is slidably mounted on piercing device 96. For example, inserter 82 may comprise an inserter passage 106 (FIG. 6) extending along the entire inserter length 108 wherein inserter passage 106 is sized to receive piercing device 96 therethrough. Preferably, inserter passage 106 is sized to freely slidably receive piercing device 96 therethrough. Piercing device length 102 (FIG. 7) may be larger than inserter length 108 (FIG. 6) such that when piercing device 96 passes completely through inserter passage 106, piercing device 96 extends from the ends of inserter 82, as illustrated in FIG. 8.

When inserter passage 106 receives piercing device 98, perceiving device 98 can guide inserter 82 is a direction substantially parallel to piercing length 98. For example, piercing device 98 can be used to guide inserter 82 toward and at least partially through a tissue incision.

A further embodiment relates to a method for inserting a cannula into tissue. In this embodiment, an inserter, preferably in combination with a piercing device are used to insert a cannula into tissue. Any cannula 10, inserter 82 and piercing device 96, as outlined above, may be used to perform the method that will now be discussed. For clarity, the same reference numerals are used to designate elements of the different embodiments that are analogous to one another. For brevity, the description of previously discussed figures is not repeated.

Outer cannula member 12 may first be provided on inserter 82. FIG. 4 illustrates outer cannula member 12 being received by inserter 82. FIG. 5 illustrates outer cannula member 12 mounted to inserter 82. Inner cannula member 14 may then be secured to inserter 82. FIG. 5 illustrates outer cannula member 12 being coupled to inserter 82. FIG. 6 illustrates inner cannula member 14 mounted to inserter 82.

Preferably, outer cannula member 12 is mounted to inserter 82 before inner cannula member 14 is mounted to inserter 82. However, the order of these steps could be reversed. For example, depending upon the configuration of hand grip portion 86, outer cannula member 12 may be placed on inserter 82 after inner cannula member 14 is secured to inserter 82. As an example, outer member 12 could be configured to pass over hand grip portion 86 if the inner cannula member 14 is mounted to inserter 82 before the outer cannula member. As a further example, outer member 12 could be mounted to inserter 82 at a location that is unaffected and unimpeded by inner cannula member 14.

Piercing device 96 may then be advanced a desired distance into tissue 42, as illustrated in FIG. 7. Typically, the piercing device will be advanced until it passes through inner tissue surface 50 or until it contacts a joint of interest. Typically, piercing device is pushed into tissue 42 using manual force.

Inserter 82 may then be placed over piercing device 96 (see for example FIG. 8). A person skilled in the art will appreciate that piercing device 96 can be positioned through inserter 82 either before or after the piercing device 96 is advanced a desired distance into tissue 42.

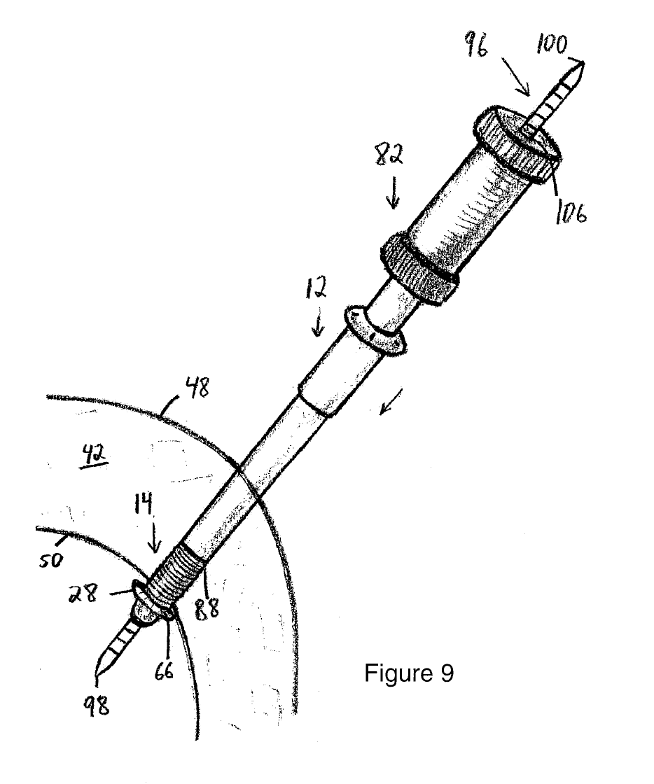

Inserter 82 may then be advanced (e.g., slid) along piercing device 96 into the tissue, as illustrated in FIG. 8. Piercing device 96 can guide inserter 82 into the tissue in a direction parallel to the piercing device length 102 (FIG. 7). Preferably, piercing device 96 remains substantially stationary while inserter 82 is advanced into tissue 42. To remain substantially stationary, piercing device first end 98 may contact a joint or other solid bodily component, while a holding or pushing force may be exerted on the piercing device second end 100. The inserter is advanced into the tissue until at least a portion of inner cannula member 14 that is located at insertion end 88 of inserter 82 passes inner tissue surface 50, as illustrated in FIG. 9. Preferably, inserter 82 is advanced until at least a portion of inner flange tissue-facing surface 66 abuts inner tissue surface 50.

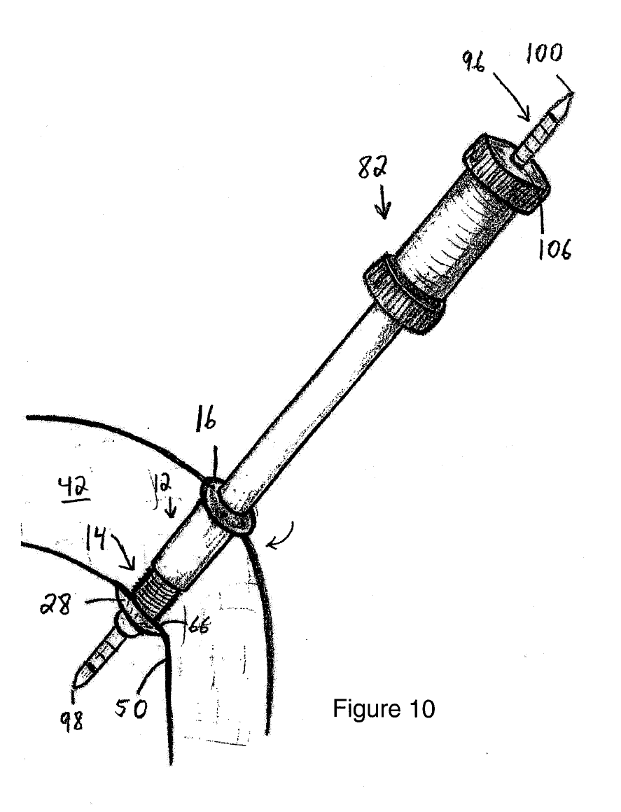

Outer cannula member 12 may then be advanced along inserter 82 toward insertion end 88. FIGS. 9 and 10 illustrate outer cannula member 12 before and after it is advanced toward insertion end 88, respectively. Outer cannula member 12 is advanced along inserter 82 until it is in close enough proximity to inner cannula member 14 to be engaged with inner cannula member 14. When the two cannula members are close enough to one another, outer cannula member 12 is coupled to inner cannula member 14 to form cannula 10. FIG. 10 illustrates outer cannula member 12 engaged with inner cannula member 14 within tissue 42.

After outer cannula member 12 is coupled to inner cannula member 14, inserter 82 may then be detached from inner cannula member 14. As a result, inserter 82 is decoupled from cannula 10. Piercing device 96 may then be removed from tissue 42

Inserter 82, and piercing device 96 if it has not yet been removed, may then be removed from tissue 42. As a result, cannula 10 is inserted in tissue 42 and provides a continuous passage 40 through the tissue, as illustrated in FIG. 3.

An advantage of this design and method is that inserting the canunla over the inserter permits a precise insertion with minimal damage to surrounding soft tissue and eliminates or at least reduces the risk of misdirecting the cannula into the tissue.

It will be appreciated that cannula 10 may be utilized by itself or in combination with inserter 82 and/or piercing device 96 as disclosed herein. In addition, the cannula 10 herein may be utilized using any inserter and/or piercing device known in the art. In addition, inserter 82 may be utilized using any two-piece cannula known in the art that has been modified to engage inserter 82. It will also be appreciated that any of the features disclosed herein may be used by themselves, or with any other feature.

What has been described above has been intended illustrative and non-limiting and it will be understood by persons skilled in the art that other variances and modifications may be made without departing from the scope of the disclosure as defined in the claims appended hereto.

1. A cannula insertable through tissue, the cannula comprising:
a) an outer cannula member that is engageable with an inner cannula member;
b) the outer cannula member having an outer portion positionable in abutting relationship with an outer tissue surface of the tissue and having an outer instrument opening, and a first shaft with a first passage aligned with the outer instrument opening; and,
c) the inner cannula member having an inner flange positionable in abutting relationship with an inner tissue surface of the tissue and having an inner instrument opening, and a second shaft with a second passage aligned with the inner instrument opening, the inner flange being fixedly mounted in position with respect to the second shaft and independently engageable in abutting relationship with the inner tissue surface when the inner and outer cannula members are engaged;
d) whereby the first and second passages define a continuous passage when the inner and outer cannula members are engaged.

2. The cannula according to claim 1, wherein the second shaft has a first end and a distal end spaced from the inner flange and the first shaft is configured to terminate at a location on the second shaft spaced from the first end of the second shaft when the inner and outer cannula members are engaged.

3. The cannula according to claim 1, wherein the second shaft is engagably received in the first shaft.

4. The cannula according to claim 3, wherein the first shaft has an inner surface having a first engagement member and the second shaft has an outer surface having a second engagement member.

5. The cannula according to claim 4, wherein the first and second engagement members comprise interengaging screw threads.

6. The cannula according to claim 1, wherein the second shaft has first end and a distal end spaced from the inner flange and the inner flange is located adjacent the first end.

7. The cannula according to claim 1, wherein the inner flange is an annular flange extending around the perimeter of the second shaft.

8. The cannula according to claim 7, wherein the inner flange has an inner flange tissue-facing surface positioned to face the inner tissue surface, the inner flange tissue-facing surface is concave.

9. The cannula according to claim 1, wherein the outer portion further comprises at least one biological matter vent spaced from the outer instrument opening.

10. The cannula according to claim 1, wherein the outer portion further comprises an outer portion tissue facing surface and the first shaft terminates proximate the outer portion tissue facing surface.

11. The cannula according to claim 1, wherein the outer portion comprises an annular flange extending around the first shaft.

12. The cannula according to 11, wherein the outer portion has an outer portion tissue facing surface that is concave.

13. The cannula according to claim 1, wherein at least one of the outer portion and the inner flange comprises a frame portion and a membrane portion and the membrane portion has increased flexibility compared to the frame portion.

14. The cannula according to claim 1, wherein the inner cannula member has an inserter-engaging member.

15. A cannula kit including a cannula insertable through tissue, the kit comprising:
a) an outer cannula member that is engageable with an inner cannula member;
b) the outer cannula member having an outer portion positionable in abutting relationship with an outer tissue surface of the tissue and having an outer instrument opening, and a first shaft with a first passage aligned with the outer instrument opening;
c) the inner cannula member having an inner flange positionable in abutting relationship with an inner tissue surface of the tissue and having an inner instrument opening, and a second shaft with a second passage aligned with the inner instrument opening, and an inserter-engaging member; and,
d) an inserter engageable with the inserter-engaging member.

16. The kit of claim 15 wherein the inserter comprises a longitudinally extending member having a hand grip portion and an insertion end spaced therefrom, the inserter has a cannula-engaging member provided on the insertion end and releasably engageable with the inner cannula member.

17. The kit according to claim 16, wherein the first shaft has an inner surface having a first engagement member and the second shaft has an outer surface having a second engagement member and an inner surface having the inserter-engaging member.

18. The kit according to claim 17, wherein the first and second engagement members comprise interengaging screw threads.

19. The kit according to claim 17, wherein the inserter-engaging member comprises a screw thread and the insertion end is threadedly engageable therewith.

20. The kit according to claim 15, wherein the second shaft has a first end and a distal end spaced from the inner flange and the inner flange is located adjacent the first end.

21. The kit according to claim 15, wherein the outer cannula member is slidably receivable on the inserter.

22. The kit according to claim 15, further comprising a piercing device for creating an incision in the tissue, and the piercing device has a plurality of insertion depth markings disposed along its length.

23. The kit according to claim 22, wherein the inserter further comprises an inserter passage sized to receive therein the piercing device.

24. The kit according to claim 15, wherein the inserter further comprises an inserter passage sized to slidably receive therein a longitudinally extending piercing device, the piercing device having a length longer than the inserter.

25. A method for inserting a cannula in a tissue comprising:
a) mounting an outer cannula member to an inserter and securing an inner cannula member to an insertion end of the inserter;
b) advancing a piercing device a desired distance into the tissue and positioning the piercing device through the inserter;
c) advancing the inserter along the piercing device into the tissue until at least a portion of the inner cannula member that is located at the insertion end of the inserter passes an inner tissue surface of the tissue;
d) advancing the outer cannula member along the inserter toward the insertion end of the inserter and coupling the outer cannula member to the inner cannula member;
e) detaching the inserter from the inner cannula member; and,
f) removing the inserter and the piercing device from the tissue.

26. The method according to claim 25, wherein the outer cannula member comprises a first shaft with a first passage aligned with an outer instrument opening, the inserter is slidably receivable in the first passage, the inner cannula member is rotatably mountable to the inserter and step (a) comprises rotatably mounting the inner cannula member to the inserter and sliding the outer cannula member onto the inserter.

27. The method according to claim 26, wherein the inserter comprises an inserter passage, the piercing member is slidably receivable in the inserter passage and step (b) comprises sliding the inserter onto the piercing device.

28. The method according to claim 27, wherein the inner and outer cannula members are rotatably mountable to each other and step (e) comprises sliding the outer cannula member along the inserter to engage the inner cannula member and then rotatably coupling the inner and outer cannula members.

29. The method according to claim 27, wherein step (f) comprises rotatably decoupling the inserter and the inner cannula member while maintaining the inner and outer cannula members in a coupled state.
